(19) United States
(12) Patent Application Publication
(10) Pub. No.: US 2006/0173263 A1
(43) Pub. Date: Aug. 3, 2006

(54) Neural interface assembly and method for making and implanting the same

(76) Inventors: Jiping He, Tempe, AZ (US); Gregory B. Raupp, Tempe, AZ (US); Stephen P. Massia, Mesa, AZ (US); Amarjit Singh, Tempe, AZ (US); Chun-Xiang Tian, Chandler, AZ (US)

Correspondence Address: NEEDLE & ROSENBERG, P.C., SUITE 1000, 999 PEACHTREE STREET, ATLANTA, GA, 30309-3915, US

(21) Appl. No.: 11/180,967

(22) Filed: Jul. 12, 2005

Related U.S. Application Data
(63) Continuation-in-part of application No. PCT/US03/38027, filed on Dec. 1, 2003.
(60) Provisional application No. 60/445,156, filed on Feb. 4, 2003.

(51) Int. Cl. A61B 5/04

(52) U.S. Cl. 600/378

(57) ABSTRACT

An implant assembly for creating a neural interface with a central nervous system having at least one biocompatible intracortical electrode is presented along with a method of making and implanting device. The mechanical, electrical and biological characteristics of the assembly support its use as a reliable long term implant.

This application is a continuation-in-part of International Application No. PCT/US2003/038027, filed Dec. 1, 2003, which claims priority to U.S. Provisional Application No. 60/445,156, filed Feb. 4, 2003; and this application claims priority to U.S. Provisional Application No. 60/______, filed Jul. 12, 2004; which applications are incorporated herein fully by this reference.

This invention was made with government support under Grant No. MDA9720010027 awarded by the Defense Advanced Research Projects Agency (DARPA), Department of Defense. The United States Government may own certain rights to this invention.

### FIELD OF THE INVENTION

The present invention relates, generally, to an assembly for creating a neural interface with the central nervous system and a method for making and implanting the same. More particularly, the present invention is directed to a device for creating a multi-channel neural interface for long-term recording or stimulation in the cerebral cortex.

### BACKGROUND OF THE INVENTION

Since the advent of the simple intracortical single microelectrode four decades ago, continued technical advances in the biological, materials and electronics fields have fueled a steady advance in the development of neural interfaces. Today, advanced devices that are available for implantation into the brain have multiple electrode sites, are chronically implantable, and can include circuitry for on-board signal processing. These complex structures are ideal for many potential clinical applications and basic research applications. For example, there is continuing evidence that a neural interface providing reliable and stable long-term implant function could be used for the realization of clinically useful cortical prostheses for the handicapped. In addition, the utility of multi-electrode arrays has already been demonstrated in basic research studies which have provided fundamental insights into parallel processing strategies during sensory coding in the brain.

Development of the first single penetrating electrode device spawned the first of generation of intracortical neural interfaces. In the first generation, microelectrodes consisted of known electrically conductive materials that were stiff enough to be inserted through either the pia or the dura membrane without buckling. These microelectrodes are still in use today and may consist of simple materials such as a stiff and sharpened insulated metallic wire or a drawn glass-pipette filled with an aqueous conductor. Because of their high impedance and small site sizes, these electrodes must be rigorously positioned near their target neurons using precision micromanipulation in order to be effective. Recordings can only be held for several minutes to several hours with these microelectrodes before repositioning is required which reduces their attractiveness for long term chronic implant.

Researchers now routinely employ multiple single microelectrodes aligned into arrays to provide ever-increasing numbers of electrode sites in one device. Some devices have positional electrodes while others have modified single electrodes (with larger site sizes and/or reduced impedances) which are capable of recording neural activity without precise positioning. These devices can remain functional upon implant for one to twelve months but the same individual neurons can not be “tracked” for longer than about six weeks.

The second generation of implantable neural interfaces includes complex electrode designs which allow for batch fabrication of multiple-site devices. These devices are usually monolithic, multi-site devices having the capability for integrated electronics and cabling, and are created by incorporating planar photolithographic and/or silicon micromachining manufacturing techniques from the electronics industry. Devices made of silicon, or devices incorporating molybdenum, which are stiff enough to penetrate the pia upon implantation have been used for recording or stimulation of the cerebral cortex. Like the first generation devices, these intracortical interfaces can remain secure in the brain for extended periods of time but recording quality and electrode yield typically diminish with time. Other devices are polyimide-based and have been designed to provide a conformal coverage when placed upon the curved surface of the brain but many of these applications require electrodes to be implanted into the cortex.

The promise of advanced neuroprosthetic systems to significantly improve the quality of life for a segment of the handicapped, such as, for example, the deaf, blind, or paralyzed population, hinges on the development of an efficacious and safe neural interface for the central nervous system. Accordingly, there is a need for a reliable, consistent, and long-term neural interface device or assembly for the central nervous system which overcomes the shortcomings of previous generation devices described above.

### SUMMARY

In one aspect, the present invention provides an assembly for neural interfacing with a subject, and methods of making and implanting the assembly. More specifically, in a further aspect, the assembly can comprise a biocompatible thin-film electrode that can be fabricated using planar photolithographic silicon processing compatible techniques.

In one aspect of the present invention, the assembly provides an end user the ability to study of the functions of neurons and their associated networks within the subject. This can include the study of the function of neurons located in a particular area of interest as well as the study of the functions of neurons located outside of the particular area of interest. In a further aspect, the assembly provides for the capture of neural signals. In an alternative aspect, the assembly of the present invention can be used to inject a signal into a neuron or neural system.

In one aspect, the present invention comprises an assembly comprising a plurality of electrodes arranged about a hub member. In one aspect, at least a portion of the electrodes are flexible. In another aspect, at least a portion of the assembly is formed of a biocompatible polymeric material. In a further aspect, the present invention comprises a guide assembly for properly implanting an electrode or an assembly of electrodes.

Additional aspects of the invention will be set forth, in part, in the detailed description, figures and any claims which follow, and in part will be derived from the detailed description, or can be learned by practice of the invention. It is to be understood that both the foregoing general description and the following detailed description are exemplary and explanatory only and are not restrictive of the invention as disclosed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows a schematic illustration of the implementation of a Brain-Machine interface.

FIG. 2 shows an exemplary intracortical electrode for implant into live tissue.

FIG. 3 shows an exemplary implant assembly of the present invention, showing a plurality of intracortical electrodes extending therefrom a hub member and a connector operably connected to the connector.

FIG. 4(a) shows a top view of an exemplary implant assembly.

FIG. 4(b) shows a cross-sectional view of the implant assembly of FIG. 4(a).

FIG. 5(a) shows an enlarged partial top view of the hub member and the electrodes of the implant assembly of FIG. 4(a).

FIG. 5(b) shows an enlarged partial side elevational view of the implant assembly of FIG. 4(a).

FIG. 5(c) shows an enlarged partial top view of one of the electrodes of the implant assembly of FIG. 4(a), showing a plurality of transponder sites and a via.

FIG. 6(a) is a microscope image of the implant assembly of FIG. 4(a).

FIG. 6(b) is a microscope image of an enlarged partial top view of the hub member and the electrodes of the implant assembly of FIG. 4(a).

FIG. 6(c) is a microscope image of an enlarged partial top view of one of the electrodes of the implant assembly of FIG. 4(a).

FIG. 7 is a top view of an exemplary electrode of the present invention, showing a plurality of transponder cites and a via.

FIG. 8 is a perspective view of an implant assembly after surgical implantation.

FIGS. 9A and 9(b) show partial top views of an exemplary implant assembly positioned in a spiral pattern to increase the electrode density of the assembly.

FIG. 10 shows an exemplary implant assembly having a smaller electrode.

FIG. 11 shows a tissue cross-section that illustrates the cytoarchitecture of the motor cortex and an exemplary penetration protocol of the intracortical electrode.

FIG. 12 is a schematic diagram of a stylized extracellular recording event.

FIG. 13 illustrates the steps involved in making an exemplary intracortical electrode.

FIG. 14 illustrates the UV-VIS spectra of BCB thin film at various stages of processing.

FIG. 15 is a schematic diagram of the BCB based intracortical electrode with an additional silicon layer underneath the electrode.

FIGS. 16(a)-16(f) are schematic diagrams showing an exemplary fabrication of the BCB based intracortical electrode of FIG. 15.

FIG. 17(a) is a perspective view of a plug of a guide assembly, showing a plurality of defined slots in the plug.

FIG. 17(b) is a top view of the plug of FIG. 17(a).

FIG. 18 is a perspective view of the plug of FIG. 17(a).

FIG. 19 is a perspective view of portions of the guide assembly, showing a manipulator in contact with an underlying guide tube that has enclosed the hub member and, at least partially, portions of the electrodes of the implant assembly, and showing the electrodes being disposed in the defined slots of the plug.

FIG. 20(a) is a top view of an alternative embodiment of a plug of a guide assembly.

FIG. 20(b) is a side, cross-sectional view of the plug fastened to a portion of a skull with bore screws.

FIG. 21 illustrates a plurality of implant assemblies that are adapted to be applied in a stackable manner, and showing a plug adapted to guide respective electrodes of the plurality of implant assemblies.

FIG. 22 shows an alternative embodiment of a plug of the guide assemblies that is adapted to guide the respective electrodes of the plurality of implant assemblies shown in FIGS. 9(a)-10.

FIG. 23 is a schematic showing an exemplary method for using the plug of the present invention.

### DETAILED DESCRIPTION OF THE INVENTION

The present invention may be understood more readily by reference to the following detailed description of preferred embodiments of the invention and to the Figures and their previous and following description.

Before the present articles, devices, assemblies and/or methods are disclosed and described, it is to be understood that this invention is not limited to the specific articles, devices, assemblies and/or methods disclosed unless otherwise specified, as such may, of course, vary. It is also to be understood that the terminology used herein is for the purpose of describing particular embodiments only and is not intended to be limiting.

As used herein, the singular forms “a,” “an” and “the” include plural referents unless the context clearly dictates otherwise. Thus, for example, reference to “an electrode” includes embodiments having two or more such electrodes unless the context clearly indicates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, a “subject” refers to any living organism having a neural system. For example, in one aspect a subject can be an animal. In one aspect the animal can be mammalian. In an alternative aspect the animal can be non-mammalian. The animal can also be a cold-blooded animal, such as a fish, a reptile, or an amphibian. Alternatively, the animal can be a warm-blooded animal, such as a human, a farm animal, a domestic animal, or even a laboratory animal. Accordingly, it should be understood that the present invention is not limited to its use in connection with any one particular subject or group of subjects.

As used herein, the “biocompatible polymer” can be any polymer suitable for neural implantation in the subject. In one aspect, the biocompatible polymer can be a photosensitive or photoimagable polymer. Suitable photosensitive polymers according to the present invention can include, without limitation, photosensitive polyimide polymers and/or photosensitive nonpolyimide polymers.

An exemplary and non-limiting nonpolyimide polymers can be any polymer selected from the class of photosensitive benzocyclobutene (BCB) derived polymers. As will be appreciated upon practicing the present invention, the photoimagable property of a BCB polymer can, in one aspect, make it suitable for the manufacture of microelectronic devices. A Benzocyclobutene (BCB) biopolymer can also provide both flexibility for micro-motion compliance between brain tissues and skull and stiffness for better surgical handling. Moreover, a BCB polymer can also remain stable during relatively long-term implant functions, because it can have flexibility, biocompatibility, relatively low moisture uptake (>0.2 wt %), and a relatively low dielectric constant (˜2.6). For enhanced operation during surgical insertion, a silicon backbone layer can, if desired, also be attached to a desired region of the polymer to increase the desired stiffness. Further, BCB polymers are considered non-toxic to the fibroblast and glial cells, further enhancing their use in connection with an implantable neural interface.

Exemplary photosensitive BCB polymers that are suitable for use with the present invention can, for example, be any one of the Cyclotene® series of nonpolyimide photoimageable, polymer products available from the Dow Chemical Company. To this end, in one aspect, the BCB polymer can be Dow's Cylotene® 4026 BCB polymer.

The present invention can be used for research as well as the treatment of disease and other medical conditions. It is contemplated that the assemblies of the present invention can be appropriately sized and shaped for use in the nervous system of various subjects, including, for example, rats, mice, monkeys, and humans. Various diseases can be studied and treated by using embodiments of the invention, such as, for example, central nervous system disorders such as spinal cord trauma, brain injury, Parkinson's disease, multiple sclerosis, demylinating diseases, nerve damage, Alzheimer disease, epilepsy, and depression.

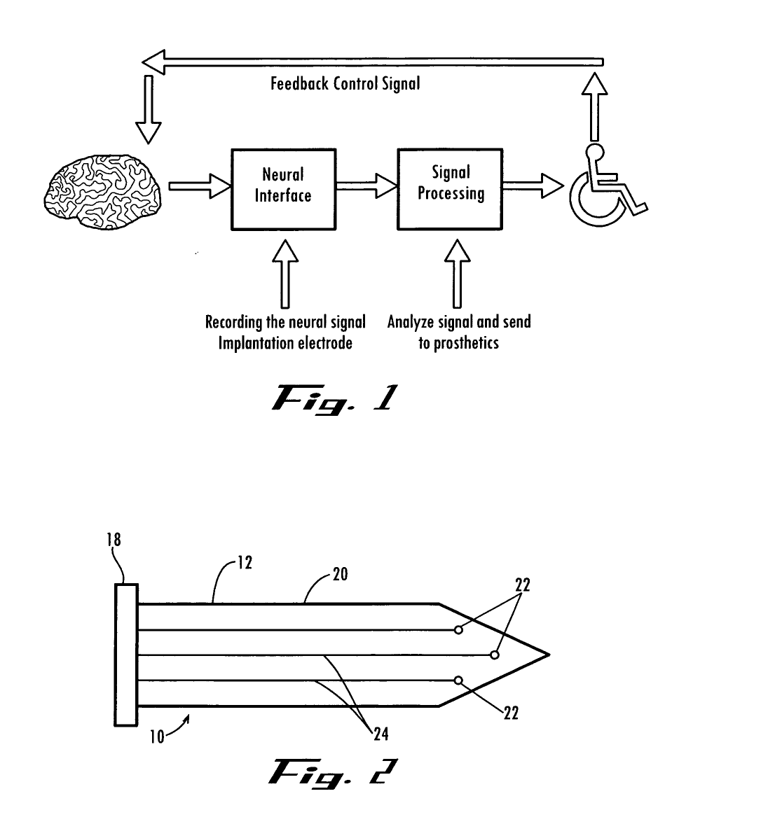

Multi-electrode arrays, which would include embodiments of this invention, provide fundamental insights into parallel processing strategies during sensory coding in the brain. In addition researchers have struggled to understand the central nervous system and develop treatments for neural impaired patients for centuries. The Brain-Machine Interface (BMI) is an emerging cutting-edge technology for studying the function of central nervous system (CNS) and therefore, facilitates the treatments of patient with neural impairment. Neuron communication is the core of nervous system activity, and understanding its signal leads to understanding how the nervous system works. Many neuroscientists therefore wish to record these neural signals in real time and from great numbers of neurons. The neural interface is a component on those types of studies. The recording of neural signal(s) by a neural interface is a technique for studying the central nervous system. To aid the handicapped, the neural interface acquires the neural signal from the brain and drives devices, such as a wheelchair or robotic arm, to help a paralyzed patient to finish basic activities or tasks in everyday living. An embodiment of that system is shown in block diagram form in FIG. 1. The whole system shown in FIG. 1 is called Brain-Machine Interface. The neural interface is a component in such a system because it is the bridge of the brain and the machine (wheelchair or robotic arm).

The term “neural interface” can include all of the elements of a system between the central processor of the computer and the nervous system tissue as used in neuron-technology—that is, from the data-acquisition interface circuitry; through the wireless electromagnetic link that couples outer world and inner body, the internal wires and electrode tips, and the subsequent tissue volume conductors; to the final target: a whole nerve, a fascicle, an axon, or a soma. See, W. L. Rutten, H. J. van Wier, and J. H. Put, “Sensitivity and selectivity of intraneural stimulation using a silicon electrode array,” IEEE Trans Biomed Eng, vol. 38, pp. 192-8, 1991. Embodiments of the present invention are an integral part of that neural interface and can acquire neural signals and/or can inject signals into the neural system.

The neural signal is called an “action potential”, which transports the information among the neurons. A neural interface providing reliable and stable long-term implant function can be used for the realization of clinically useful cortical prostheses for the blind. See, G. Shahaf and S. Marom, “Learning in networks of cortical neurons,” J Neurosci, vol. 21, pp. 8782-8, 2001; and G. S. Brindley and W. S. Lewin, “The sensations produced by electrical stimulation of the visual cortex,” J Physiol, vol. 196, pp. 479-93, 1968.

As briefly summarized above, in one aspect the present invention provides an assembly 10 for neural interfacing with a subject, and methods of making and implanting the assembly. More specifically, in a further aspect, the assembly 10 can comprise a biocompatible thin-film intracortical electrode 12. In one aspect, the assembly can be fabricated using planar photolithographic silicon processing compatible techniques. In another aspect, at least a portion of the electrode 12 can be formed from a biocompatible polymer, such as, for example and not meant to be limiting, benzocyclobutene (BCB), a polymer with low dielectric constant, low electrical loss factor at high frequencies, low moisture absorption, low cure temperature, high degree of planarization due to the low viscosity, low level of ionic contaminant, optical clarity, good thermal stability, chemical resistance and biocompatibility.

As will be described in more detail below, the design of the assembly 10 provides an end user the ability to study of the functions of neurons and their associated networks within the subject. This can include the study of the function of neurons located within a particular volume or area of interest as well as the study of the functions of neurons located outside of the particular volume or area of interest. In alternative aspects, the assembly provides for the capture of neural signals of the subject and/or the injection of a signal into a neuron or neural system of the subject.

One embodiment of the invention can comprise an assembly 10 comprising a single intracortical electrode 12. Alternative embodiments provide an assembly 10 comprising a plurality of intracortical electrodes 12 arranged about a hub member. In one aspect, at least a portion of each intracortical electrode is flexible. In another aspect, at least a portion of the assembly is formed of a biocompatible polymeric material. In a further aspect, the present invention comprises a guide assembly for properly implanting the electrode(s).

In one aspect, the assembly 10 comprises a thin film substrate 20 that is at least partially formed by a biocompatible polymer, such as, for example and not meant to be limiting, benzocyclobutene (BCB). As will be described in more detail below, at least one transducer site 22 is formed in the thin film substrate for sensing and converting biophysical phenomenon to an electrical signal. A separate conductor 24 is electrically coupled to each transducer site and routed along the thin film substrate for transmitting the electrical signal. The transducer site 22 could also be a means for injecting a signal into the living tissue. One would appreciate that it is contemplated that the transducer site could act as either or both of a sensing means and an injecting means.

In embodiments of the invention comprising multiple intracortical electrodes 12, each intracortical electrode can provide recording or stimulation of multiple depths in the brain. The intracortical electrodes 12 have an elongate length and the transponder sites are positioned predetermined distance(s) from the distal end 18 of the intracortical electrode. It is contemplated that the transponder sites can be positioned on an inner face of the intracortical electrode or an outer face of the intracortical electrode. Alternatively, transponder sites 22 can be positioned on both or the inner and outer faces of the intracortical electrode. In a further aspect, at least one intracortical electrode 12 of the assembly 10 can have transponder sites that are positioned at different locations from the other electrodes. In a further aspect, the transponder sites can be positioned adjacent the tip 18 of the intracortical electrode 12.

Thus, the implant assemblies 10 of the present invention can be positioned such that the transponder sites 22 face “outside” (i.e., transponders on the outer face of the intracortical electrode) of a predetermine volume or area so that the neurons surrounding the volume or area are monitored or stimulated, or “inside” (i.e., transponders on the inner face of the intracortical electrode) so that the neurons clustered in the predetermined volume or area are monitored or stimulated. By combining several of the electrode assemblies together one can design experiments to investigate a wide variety of basic neuroscience and clinical neurotrauma or degenerative disease issues in the central nervous system, including the spinal cord.

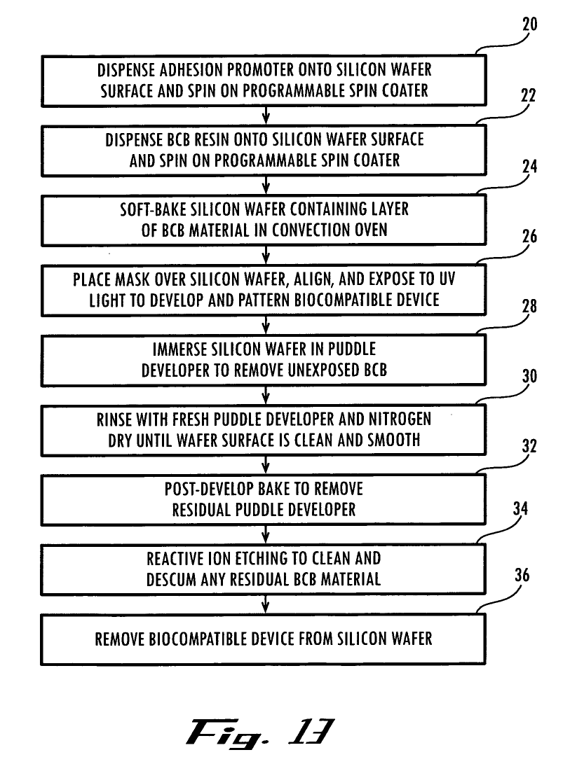

To help handicap patients, the motor cortex is of interest because the motor cortex controls one's movement. FIG. 11 shows the typical cytoarchitecture of the motor cortex with the different layers. Molecular layer (layer I), Outer Granular layer (layer II), and Pyramidal layer (layer III) perform most of the intracortical association functions, with especially large numbers of neurons in layer II and III making short horizontal connections with adjacent cortical areas. The most incoming specific sensory signals terminate in the Inner Gramular layer (layer IV). Most of the output signals leave the cortex from neurons located in Ganglionic and Multiform layers (layer V and VI) and the very large fibers to the brain stem and cord arise generally in layer V.

In one embodiment, the intracortical electrode 12 of the present invention targets layer V (Ganlionic layer). FIG. 11 shows an exemplified penetration protocol of the intracortical electrode of the present invention, with the transducer sites 12 proximate the tip 18 of the electrode 12 being positioned in layer V to record the output neuronal signals leaving the cortex. The distance between the surface of the cortex and the layer V is usually 2 mm, so in one embodiment of the invention, the insertion portion of the electrode is designed to be about 1.5 to about 2.5 mm long, including additional insertion portion lengths of approximately 1.6 mm, 1.7 mm, 1.8 mm, 1.9 mm, 2.0 mm, 2.1 mm, 2.2 mm, 2.3 mm, and 2.4 mm, such that, in use, the transducer sites of the intracortical electrode 12 are positioned therein layer V to record the neuron signal more effectively.

As noted above, embodiments of the present invention can comprise Benzocyclobutene (BCB), which in its base form is a polymer liquid or resin available under the tradename of Cyclotene 4026. The resin contains 46 wt % B-staged divinylsiloxane-bis-benzocyclobutene in a mesitylene carrier solvent, along with trace amounts of polymerized 1,2-dihydro-2,2,4-trimethylquinoline, 2,6-bis{(4-azidophenyl)methylene}-4-ethylcyclohexanone, and 1-1′-(1-methylethylidene)bis{4-(4-azidophenoxy)benzene}. BCB is a photosensitive, colorless, and high viscosity material.

BCB can be converted to and used as a biocompatible material in many applications such as biosensors, catheters, pacemakers, tissue replacement, medication dispensers, and other medical devices implanted in the body. BCB has many desirable electro-physical-chemical properties, including bio-compatibility and reliability for implantable devices. In order to assess and confirm the effectiveness of BCB as a biocompatible material, a number of studies and tests have been performed. The cytotoxicity and cell adhesion behavior of Cyclotene 4026 coatings exposed to monolayers of glial and fibroblast cells in vitro has been evaluated. The studies have confirmed BCB films deposited on silicon wafers using micro-fabrication processes have not adversely affected standard tests such as 3T3 fibroblast and T98-G glial cell function in vitro.

In FIG. 2, an exemplary intracortical electrode 12 of an implant assembly is shown. The intracortical electrode comprises a flexible substrate 20 that is suitable for implantation into living tissue. In one example, at least a portion of the flexible substrate comprises a biocompatible polymer, such as the exemplified BCB material. The transponder sites 22 are positioned on the substrate 20 and are constructed and arranged to convert electro-chemical and physical reactions and biophysical phenomena present in the living tissue to electrical signals. The conductors 24, which can be formed of metal material, are disposed in the substrate 20 to route the electrical signals from the transponder sites 22 to a connector 18. It is contemplated that the transducer sites can be formed integrally with the conductors, that is, a portion of the conductor can form the transducer site. The metal conductor 24 may be disposed on the surface of substrate 20, or sandwiched between first and second layers of substrate 20. The connector 18 provides an interface to other conductors to transmit the electrical signals to remote measurement instrumentation (not shown). The intracortical electrode 12 is adapted for insertion into living tissue, for example, as a neural implant.

One embodiment of the present invention is a method of making a biocompatible assembly 10 comprising the steps of dispensing a biocompatible polymer, such as the exemplified benzocyclobutene (BCB) resin, onto a silicon wafer, spinning the silicon wafer to distribute the polymer, curing the polymer on the silicon wafer to form a polymeric thin film layer, patterning the biocompatible assembly in the polymeric thin film layer on the silicon wafer, removing residue from the silicon wafer, performing a final cure of the polymeric thin film layer, and removing the biocompatible assembly from the silicon wafer.

In another embodiment, the present invention is a biocompatible assembly 10 comprising a substrate 20 including benzocyclobutene material which is suitable for implant into living tissue. Another embodiment of the invention provides a method of using benzocyclobutene material in a biocompatible assembly, comprising the step of forming a substrate from the benzocyclobutene material so that the substrate is suitable for implant into living tissue.

FIG. 13 illustrates the steps of making the intracortical electrode noted above from the exemplified BCB resin which can be implanted in vitro. Photosensitive BCB resin stored at −20° C. in a light-proof container will maintain a shelf life of about one year. At 4° C., the shelf life of BCB is reduced to one or two months, and at room temperature the shelf life is only one or two weeks.

Processing is performed in a class 100 clean room. A 15 milliliter (mL) dropper bottle is pre-rinsed in distilled water to remove particles and then allowed to dry. Fresh BCB is taken from the −20° C. stock and transferred to the clean dropper bottle. BCB resin is allowed to equilibrate to room temperature for at least 3 hours before use. A 4″ diameter silicon wafer or other suitable substrate is selected for application of BCB. The silicon wafer is cleaned in a reactive ion etcher for about 5 minutes at about 50 watts in about 50 standard cubic centimeter/meter (sccm) oxygen flow at about 100 millitorr total pressure to remove organic contaminants.

After cleaning, the silicon wafer is placed into a programmable spin coater and an adhesion promoter is dispensed onto the middle of the wafer surface to promote adhesion of the BCB resin, as described in step 20. The adhesion promoter contains greater than 98 wt % 1-methoxy-2-propanol, less than 1 wt % water, and other trace elements. The programmable spin coater is fitted with a 2″ diameter vacuum chuck to reduce backside contamination. The bowl is lined with cleanroom wipes along the bottom and sides in order to make it easier to keep the bowl clean and further to attenuate wind currents inside the bowl and to catch any solidified BCB strands that form during spinning.

For adhesion promoter application, the bowl cover is left off to facilitate evaporation of the adhesion promoter solvent. Enough adhesion promoter is applied to cover the entire wafer surface, typically about 1-5 mL for the 4″ silicon wafer. The spin coater is spun at about 800 rpm for about 30 seconds to distribute the adhesion promoter over the wafer surface, followed by a linear ramp to about 2000 rpm over about 10 seconds. The wafer is dried at about 2000 rpm for about 30 seconds to thin the adhesion promoter to the desired thickness. The spin coater is spun down to zero in about 10 seconds.

After adhesion promoter application, about 1-5 mL of BCB resin is dispensed from the dropper bottle onto the center of the silicon wafer surface, as described in step 22. The spin coater ramps up to about 800 rpm over about 10 seconds and spins at about 800 rpm for about 10 seconds. Subsequently, it ramps up to 2000 rpm in 10 seconds and spins at 2000 rpm for 30 seconds to distribute an even thin layer of film. The spin coater ramps down to stop in about 10 seconds. The resulting BCB thin film distributes evenly over the wafer surface with a thickness of about 13 micrometers.

The spin coater bowl cover must be in place during spin coating of the BCB to keep the bowl saturated with mesitylene vapor and to retard the formation of solidified BCB strands. These strands are formed when mesitylene rapidly evaporates from the BCB resin. The strands have an appearance similar to spun sugar or spider webs and tend to contaminate the wafer if they should flop back onto the wafer surface after being formed at the periphery of the chuck. In addition, the bowl cover alters the velocity profile of the atmosphere inside the bowl, which redirects any solidified BCB strands that form away from the wafer surface.

In some applications, the thickness of the BCB thin film layer is controlled by the amount of BCB resin dispensed onto the wafer surface or by controlling the spin rate and duration of the programmable spin coater.

Alternatively, a second layer of BCB material is formed on the first layer of BCB material for additional thickness in the resulting BCB thin film material. The second layer is formed as described for the first layer of BCB material, i.e., by dispensing an adhesion promoter, spinning the wafer to evenly distribute the adhesion promoter, dispensing BCB resin, and spinning the wafer to evenly distribute the BCB resin. It is contemplated that the metal conductors 16 can be routed between the first and second BCB layers.

The wafer containing the thin film layer of BCB material is removed from the spin coater with wafer tongs and allowed to cool to room temperature before placing in a convection oven to soft-bake, as per step 24. The spinner chuck may be cleaned with an acetone-soaked cleanroom wipe. The convection oven containing the silicon wafer is heated to about 70 to 80° C. and purged with tri-nitrogen to soft-bake the wafer for about 20 minutes. The soft-bake process removes residual mesitylene. After the soft-bake process, the thin film layer of BCB material, as prepared on the silicon wafer surface, is about 10 um in thickness.

The post-soft-bake silicon wafer is cooled to room temperature for about 5 minutes and then loaded onto a contact aligner. The contact aligner uses a photolithographic process to form the biocompatible device in the thin film layer of BCB material. In the present example, a mask having the form of a plurality of intracortical electrodes is placed in the contact aligner over the silicon wafer, as per step 26. The contact aligner uses a 350-watt mercury arc lamp with G-line (436 nm), H-line (405 nm), and I-line (356 nm) wavelengths. The exposure reliability is about 3%. Depending on whether the first or second layer of BCB is applied, the appropriate dark-field emulsion mask is loaded into the contact aligner and the silicon wafer is aligned to the mask alignment structures. The gap between the top surface of the wafer and the underside of the mask is adjusted during loading to maintain a just-contact position during the exposure so that lateral UV light scattering does not occur. The BCB-coated wafers are exposed using all three wavelengths, i.e., H-line, I-line, and G-line, with the power intensity measured at the I-line wavelength. An optical filter is attached to perform a broadband exposure and provide a good patterning of the BCB thin films. The calculation of the recommended time of exposure is based on delivering an exposure dose of 60 millijoules/CM²/PM to the BCB thin film as measured at the I-line wavelength. Since the power intensity measurement is based on H-line radiation, the time-of-exposure calculation may need to be modified slightly to account for the wavelength-dependent power reading. For example, an exposure time of 3 minutes with power densities 4.0-4.5 mW/cm² should be sufficient to obtain the desired development of 10 μm post-soft-bake BCB thin film material and patterning of the plurality of intracortical electrodes.

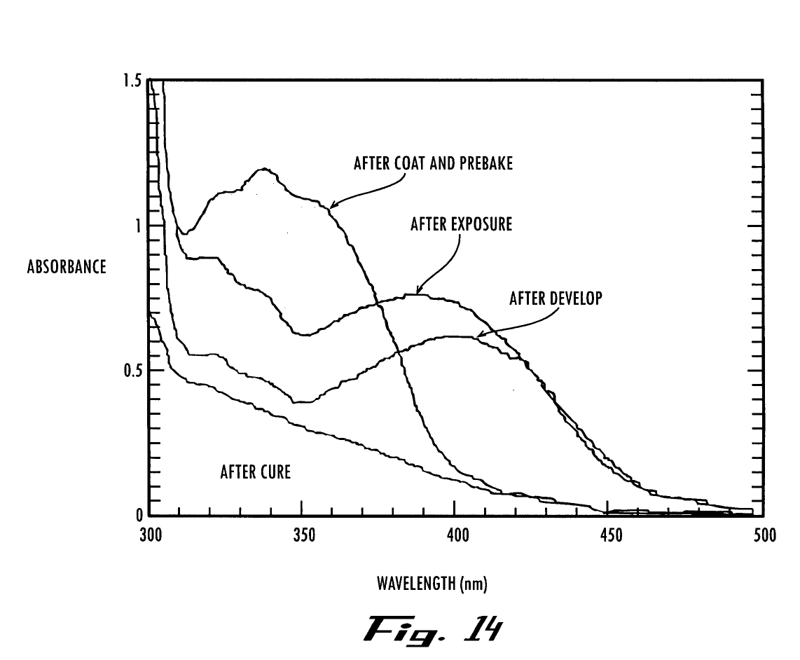

In FIG. 14, the UV-VIS spectra of BCB thin film at various stages of processing is shown. At 405 nm (H-line), the post-soft-bake BCB thin film is nearly transparent to the radiation. The H-line wavelength alone would most likely result in transmission of the radiation all the way through the thin film to the wafer surface, where it can reflect into the areas under the mask intended to be shielded from the radiation. At the 365 nm I-line wavelength, the thin film has a much higher absorbance. The I-line wavelength results in much less reflection of the H-line radiation off the underlying wafer surface, as much more cross-linking of the photosensitizers in the thin film occur during a 3-minute exposure versus similar exposure of only H-line radiation. One skilled in the art will appreciate that the thin film absorbance increases over the course of the exposure from an initially low value at the H-line wavelength to a value comparable to the final I-line wavelength value. At the I-line wavelength, the initial absorbance is high, but decreases to a final value that is still much higher than the initial H-line wavelength value.

Following UV-exposure, the silicon wafer is placed into a 10 cm diameter by 8 cm tall glass container and about 5 mL of room temperature puddle developer is added, sufficient to cover the surface of the wafer, as per step 28. The unexposed BCB material is dissolved by the puddle developer. An endpoint, defined as the time to dissolve through the entire layer of unexposed BCB material, is observed by the disappearance of a colored interference fringe pattern. For 10 μm post-soft-bake BCB thin film material, the endpoint varies from about 1 minute 20 seconds to 2 minutes. The variation is likely due to the temperature variation of the soft-bake, with hotter soft-bake temperatures leading to longer observed endpoints. Development continues an additional approximately 30% to 100% after observing the endpoint. For example, approximately 50% past a 1:30 endpoint gives a 2:15 total develop time.

After puddle development, the silicon wafer is rinsed for 10 seconds in another beaker with 5 mL of fresh, clean puddle developer, as per step 30. The silicon wafer is then immediately dried with a stream of dry nitrogen. Additional rinses in fresh puddle developer may be required to produce a clean and smooth wafer surface. The silicon wafer is baked again in the convention oven at about 75° C. for about 60 seconds to remove residual puddle developer.

The silicon wafer with the developed and patterned BCB material undergoes a final cure process to create a BCB polymer structure, as per step 32. The silicon wafer is placed in a furnace. The furnace is purged with nitrogen at room temperature for one hour to remove any residual oxygen, which is necessary to prevent oxidation of the BCB thin film during curing. After the one-hour purge, the silicon wafer is cured in the inert atmosphere by rapidly raising the temperature to about 210° C. for about 40 minutes as a partial cure for the first BCB layer. The cure temperature and time are about 250° C. for about 60 minutes for full cure of the second BCB layer, if applicable. After the required cure time, the furnace is turned off and the silicon wafer is cooled for several hours to room temperature in the inert atmosphere.

During the final cure process, a thermally activated cyclobutene ring opening occurs in the BCB monomer. The reaction forms an o-quinodimethane intermediate, which serves as the diene. The intermediate reacts with one of the many dieneophiles, i.e., a single double bond, in the BCB thin film material, and a highly cross-linked tetrahydronaphthalene structure is formed as the final product. Because there are no gaseous products formed in the BCB thin film during final curing, the BCB material can be cured as rapidly as desired without delamination concerns.

The silicon wafer is processed in a reactive ion etching chamber to clean and descum any residual BCB material, as per step 34. Partially-cured BCB thin films, which are softer and less resistant to the plasma than fully-cured thin films, are descummed with an 80:20 mixture of O₂ and CF₄ at about 100 millitorr and about 50 watts for about 5 minutes. The harder and more plasma-resistant fully cured thin films are etched for about 8 minutes using the same parameters. The silicon wafer is removed from the plasma chamber and visually inspected under a microscope. The reactive ion etching process is repeated until the residue is removed or until a dense series of nearly black spots appear on the BCB thin film. The black spots are pillars or pins of SiF or F+ metal that act as an etch mask.

In some applications, cleanly-developed BCB thin films, in particular, fully opened vias and transponder sites, could not be achieved with only H-line exposure, even with the post-develop plasma descum. For this reason, an extra processing step is performed to clean the vias in one layer BCB or recording sites in two-layer BCB. A photoresist is applied to the silicon wafer using a manual spinner at about 4000 rpm for about 30 seconds with rapid acceleration/deceleration. The photoresist film is soft-baked in a nitrogen purged convection oven for about 10 minutes at about 80° C., followed by a 5-minute cool-down period. The wafer is then exposed on the contact aligner for about 3 minutes using the complementary light-field mask.

The exposed thin film is developed for about 2 minutes 20 seconds in a deionized H₂O developer solution at room temperature. After development the patterned photoresist is hard-baked at about 80° C. for about 10 minutes. After applying the soft mask, the wafer is treated in an 80:20 O₂/CF₄ plasma in reactive ion etch mode using at about 100 millitorr and about 100 watts for about 5 minutes. A typical DC bias of 250 volts and a reflected power of 5 watts are used during this process step.

In forming the metal conductors 16, metallic traces are added to the planar electrode structure after the first BCB layer is applied. The metal traces are composed of a layer of chromium, about 20 nm in depth, followed by a layer of gold, about 200 nm in depth. The process flow for adding these metal traces includes depositing chromium followed by gold, patterning with photoresist, etching away the gold, then chromium, and finally stripping away the photoresist. Conformal layers of chromium and gold are deposited using a thermal evaporator. In step 36, the plurality of intracortical electrodes are removed from the silicon wafer.

The BCB material forming the substrate of the assembly helps ensure the biocompatibility and reliability of the assembly when implanted in vitro. The BCB material is suitable for implant in living tissue because it has flexibility, biocompability, a high degree of planarization, and low dielectric constant. The BCB material exhibits low moisture absorption and reduces bacteria infection.

To confirm the biocompatibility of BCB, the processed BCB thin films are subject to cytotoxicity and cell adhesion tests. Prior to cytotoxicity and cell adhesion tests, BCB covered silicon wafers are cleaned by (a) immersing in acetone in ultrasonic bath for two minutes, (b) rinsing with 95% ethanol and immersing in 95% ethanol in an ultrasonic bath for 20 minutes, (c) rinsing with deionized water (DI), immersing in a detergent solution in an ultrasonic bath for 20 minutes, and (d) extensively rinsing with deionized water, with one final immersion under ultrasound for 15 minutes. The wafers are placed on a sheet of aluminum foil and dried under the cell culture sterilized hood overnight. The wafers are wrapped in the same aluminum foil and autoclaved for about half an hour at 100° C.

All solutions utilized for dextran coating are filter sterilized. Dextran is immobilized to BCB thin films to modulate cell adhesion. Aminated BCB surfaces are prepared by immersion in 0.01% aqueous Poly-L-Lysine (PLL) solution and incubated overnight. Periodate-oxidized dextran is dissolved in 0.2 M sodium phosphate buffer. Immediately following surface amination procedures, oxidized dextran solution of 2 mL is added to sterile six-well multi-well dishes containing surface-aminated substrates. The substrates are allowed to incubate at room temperature for 16 hours on a rocker platform which is protected from light. Following incubation, the reaction mixture is decanted from the culture wells, and replaced by fresh 0.1M solution of sodium borohydride (NaBH₄) to reduce Schiff bases formed and to quench any free unreacted aldehyde groups present on the oxidized dextran chain. The substrates are allowed to incubate for 2 hours on the rocker platform. The NaBH₄ solution is then decanted and the substrates are rinsed gently several times with deionized water to remove unbound dextran.

The 6-well culture plates are initially coated with a 0.5% pHEMA in 95% ethanol solution to reduce cell attachment to well surfaces. Following thorough air drying of pHEMA-coated culture plates under the sterile hood, cleaned and sterile BCB materials are placed in each well. Approximately 2 mL of cell suspension in media with 15,000 cells/ml are added to each well of the culture dish. The cultures plates are then incubated at 37° C., 5% CO₂ for 24 hours.

Glial cell and fibroblast cytotoxicity are evaluated using a Live/Dead Viability/Cytotoxicity Kit. Cells are seeded into BCB material wells. Stained BCB material is examined at 100× magnification via epi-fluorescence microscope to visualize both viable fluorescein filter set and non-viable rhodamine filter set cells. The percentage of the image covered by live cells is calculated using image analysis software. The percentage values from the independent experiment are compared between each run, and then combined. The groups of BCB materials are compared between each other.

The 3T3 and T98-G cells are seeded into BCB material wells and incubated for 24 hrs. Following incubation, samples are fixed in 3.8% formaldehyde in PBS for 5 min and stained with 0.1% aqueous toluidine blue for 5 min. Stained cells are examined using phase contrast or stereomicroscopy at 100× magnification. Three random 100× fields are selected for each substrate for analysis. The extent of cell adhesion is determined for each captured digital image by calculating a percentage of cell area coverage using digital image analysis software. Final data is presented as a percentage of control adhesion. The percentage of control cell area is calculated by multiplying the ratio of % area coverage on all substrates to % cell area coverage on tissue culture plastic. The average percentage of control adhesion is determined from duplicate independent experiments.

The percent viability values for glial cells and fibroblasts cultured on BCB-coated substrates are calculated from experimental data that is collected using the cytotoxicity assay. The results indicate that 3T3 and T98-G cell viability is not significantly different from positive control values, i.e., p<0.05. Thus, BCB thin film is considered non-toxic for cultured glial cells and fibroblasts.

Cell adhesion and spreading is determined on all substrates and expressed as a percentage of control cell area coverage on tissue culture plastic reference-substrate. Morphology of adherent 3T3 fibroblasts on BCB films is similar to cells routinely cultured on tissue culture plastic. The 3T3 cell adhesion and spreading on BCB substrates is also comparable to tissue culture plastic. These results further indicate that BCB thin films do not adversely affect 3T3 fibroblast adhesion, spreading, and function in comparison to normal culture conditions on tissue culture plastic. Surface immobilization of dextran on BCB thin films significantly reduced 3T3 cell adhesion and spreading, i.e., p<0.001.

Morphology of adherent T98-G glial cells on BCB films is similar to cells routinely cultured on tissue culture plastic. T98-G cell adhesion and spreading on BCB substrates is also comparable to tissue culture plastic. These results further indicate that BCB films do not adversely affect T98-G glial cell adhesion, spreading, and function in comparison to normal culture conditions on tissue culture plastic. Surface immobilization of dextran on BCB films significantly reduced T98-G cell adhesion and spreading, i.e., p<0.001.

The study of the cytotoxicity of BCB films on silicon wafers supports the use of BCB material for microelectronic neural implant applications. The methods utilized to deposit BCB films on silicon wafers are directly applicable to processes for the microfabrication of prototype BCB-based microelectrode neural implants.

The fibroblast and glial cell lines are representative of cells that are encountered in the neural implant environment. From these cell viability and adhesion studies, it can be concluded that BCB films do not adversely affect 3T3 fibroblast and T98-G glial cell function in vitro. The BCB thin films are non-adhesive with surface immobilized dextran using methods developed for other biomaterials and applications. These results demonstrate that BCB thin films can be used for dextran-based bioactive, cell-selective coatings.

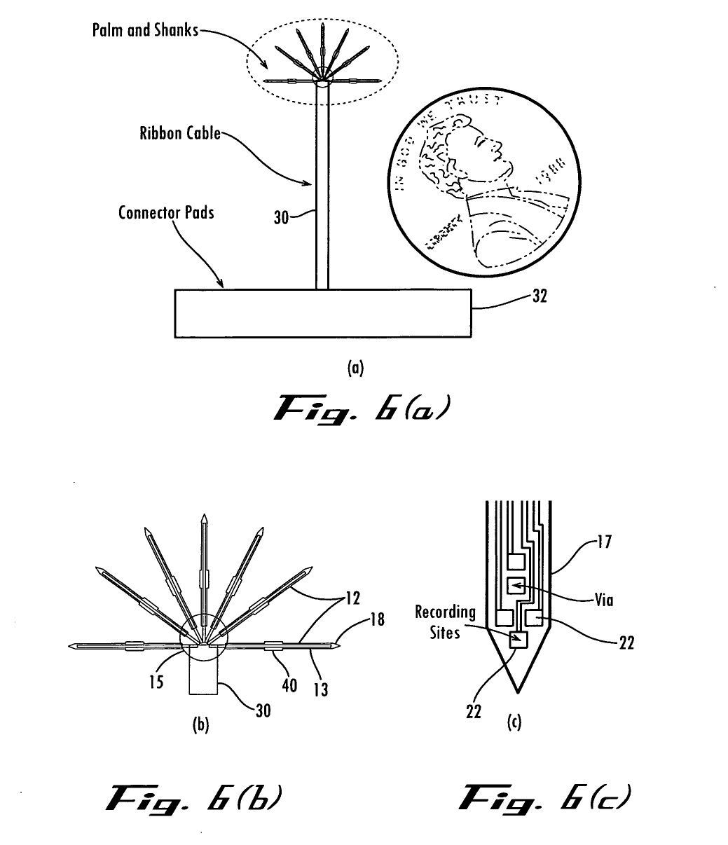

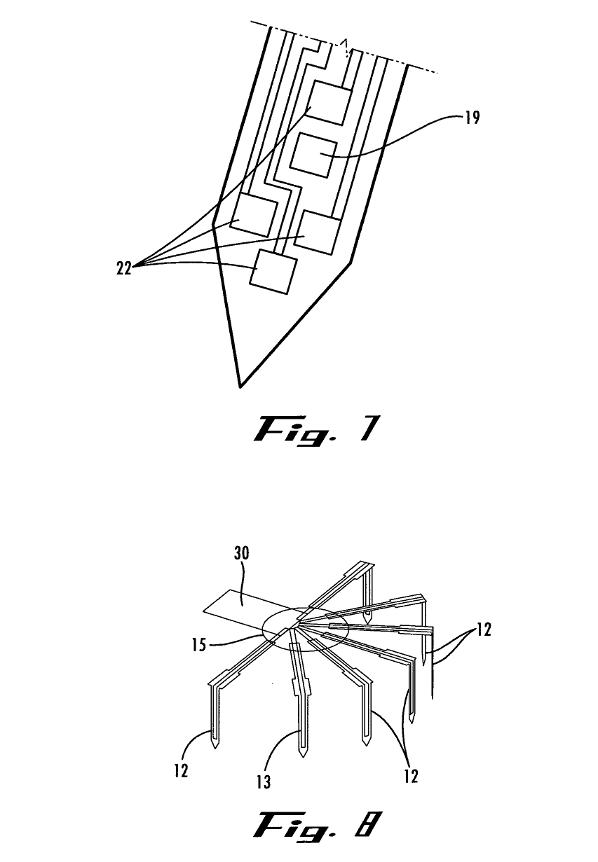

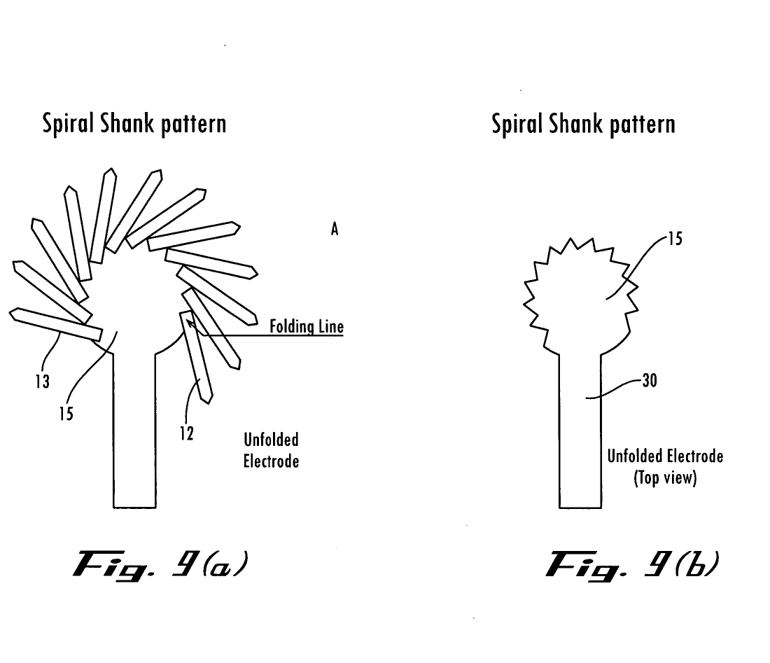

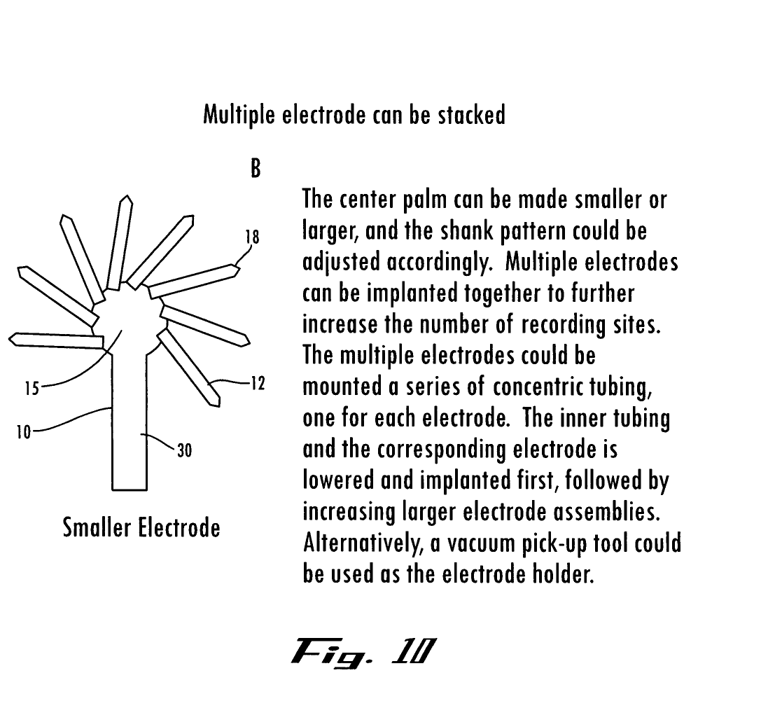

Assemblies 10 of multiple intracortical electrodes 12 can be fabricated using the same or similar techniques as described above. These multiple electrode assemblies 10 can be formed in various shapes and sizes, as suitable for the intended application or uses. FIGS. 3-10 show embodiments of a multiple electrode assembly 10, comprising three general sections: a hub member 15; at least one intracortical electrode 12 extending therefrom the hub member, and a connector 30 operable coupled to electrical conductors of the hub member.

In one aspect, the assembly can be a three-layer composite structure. However, additional layers are envisioned in other embodiments of the invention. Between two mechanically flexible and electrically insulating layers of polymer material, such as, for example, polyimide and BCB, are a plurality of electrical conductors 24 or traces formed of conductive material that are insulated from each other. On the insertion portion 13 of each of the intracortical electrodes, one or more transducer sites 22 are formed where the insulating/packaging polymer material has been removed from one layer of the polymer material to expose a portion of the underlying electrical trace 24. One or more additional layers of conductive material can be deposited on the transducer site 22 to increase the conductive surface for desired conductivity. As one will appreciate, for each transducer site, there is an electrical conductor circuit that runs from the transducer site through the length of the intracortical electrode 12, through the hub member 15, and through the connector 30. Each transducer site's electrical trace is electrically insulated from any other transducer site's electrical trace.

In an embodiment of the invention, as shown in the figures, the elongate intracortical electrode 12 can comprise a plurality of transducer sites 22 disposed along its length, each with its own electrical trace 24. The transducer sites allow the electrode assembly to measure signals from individual neurons and ensemble of neurons. In further aspects, each intracortical electrode 12 is of a type that is suitable for use on or in tissue, of a size so as not to cause excessive tissue damage, strong enough for penetration, and not normally broken or displaced by micro-motion at and around the implant site.

In one aspect, at least a portion of each of the intracortical electrodes 12 is flexible and can be bent or adjusted relative to the hub member so that the intracortical electrode can be inserted into or about a particular tissue volume or area of interest. It is contemplated that individual intracortical electrodes 12 of the assembly can be individually positioned as needed with an implantation tool to allow for their implantation into tissue at different angles and with different facing directions of the transducer sites located on the electrodes.

In a further aspect, each intracortical electrodes 12 can further comprise at least one via 17, which are adapted to reduce the disruption of the surrounding tissue by the electrode, and to enhance the support of the electrode by the surrounding tissue. The via is a defined well on the insertion portion 13 of the electrode. In another aspect, the via 17 can act as a reservoir for depositing slow release peptides or other biologicals to stimulate nerve growth toward the electrodes or to reduce inflammation in the implanted area. In addition, intervention drugs for simulating disease conditions such as Parkinson's or Alzheimer diseases can also be deposited in the defined via.

The plurality of electrodes 12 can be positioned about the hub member 15 such that the penetration tip of the electrode 12 extends outwardly away from the hub member. In one aspect, the electrodes can extend radially from the hub member. In one aspect, at least a portion of the electrode extending inwardly from the tip of the electrode forms the insertion portion of the electrode. It is contemplated that the elongate length of the electrodes can be substantially equal or can vary such that electrodes 12 of different lengths can be used in the assembly 10.

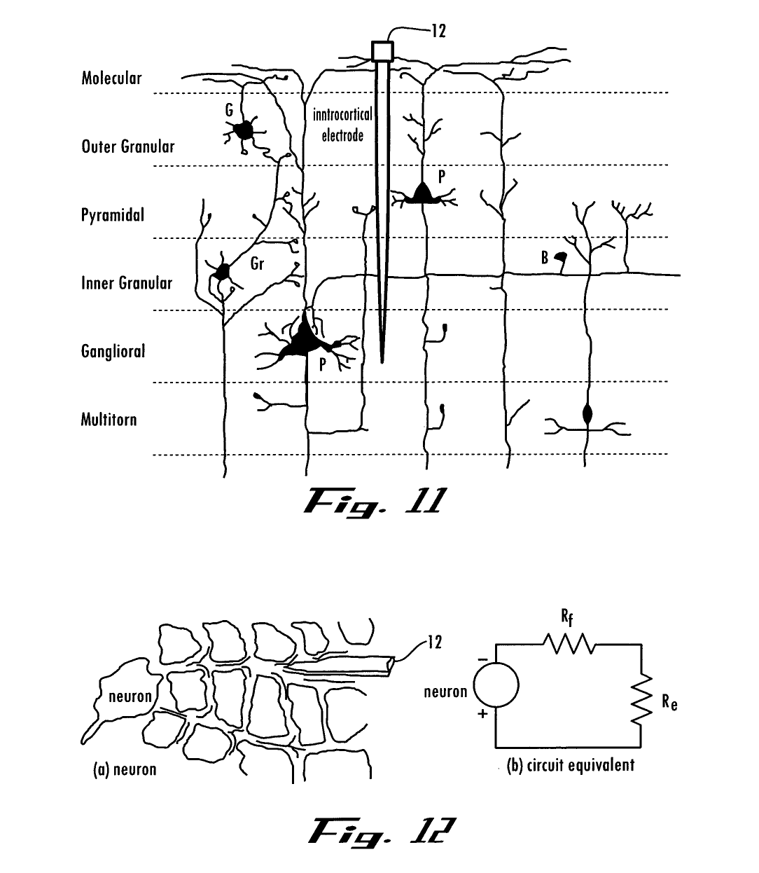

In one aspect shown in FIGS. 9 and 10, each electrode 12 is flexible at its proximal end such that the electrode can be bent at its juncture with the hub member 15. In this example, the electrode is hinged at its proximal end to the hub member. In another aspect, and as shown, the electrodes can extend non-radially outwardly from the hub member. This allows for an increased number of electrodes 12 to be formed as a part of the assembly 10.

In use, measurement of neural signals by the transducer sites takes place after implantation of the intracortical electrode 12 near a neuron. The neuron of FIG. 12 generates an action potential when its cell membrane depolarizes and ionic currents flow in the surrounding tissue. It is believed that most of the extracellular current flows in the narrow clefts between other cells (e.g., glia) present in the extra-neuronal space. See, D. A. Robinson, “The electrical properties of metal microelectrodes,” Proceedings of IEEE, vol. 56, 1968. The accompanying potential changes can be sensed if a recording electrode, such as the intracortical electrodes of the present invention, is implanted into a nearby region of relatively high current density. The transducer sites 22 of the present invention are of suitable impedance to sense the changes in extracellular current due to activities of nearby neurons. The impedance should not be too high (too much noise) or too low (cannot pick up enough charge). Preferably, the impedance of each transducer site 22 on the intracortical electrode is about 100 KΩ to about 2MΩ (measured at 1 kHz source signals). Alternatively, the impedance of each transducer site is about 200 KΩ to about 700 kΩ, more particularly about 200 KΩ to about 500 kΩ. One will appreciate that the impedance of the respective transducer sites can be adjusted by altering the size of the transducer site.

In other aspects, the individual electrodes 12 are generally rectangular in shape, but can be of different shapes for specific applications with one proximal end being physically connected to the hub member, and the distal tip end having a shape suitable for implantation on or into tissue including the brain. For example, the electrode can be pointed or otherwise shaped on the distal tip end for piercing of tissue.

In a further aspect of the intracortical electrode 12, the exterior surface of the electrode can comprise a stopping and hold mark 40 indicating the correct depth placement of the shank when it is penetrated into tissue wherein said stopping and hold mark can be a wider portion of the shank body, a ridge, a mark, or physical or visible indication of the penetration stopping point. In this example, the insertion portion 13 of the electrode extends between the stopping and hold mark and the distal tip end of the electrode.

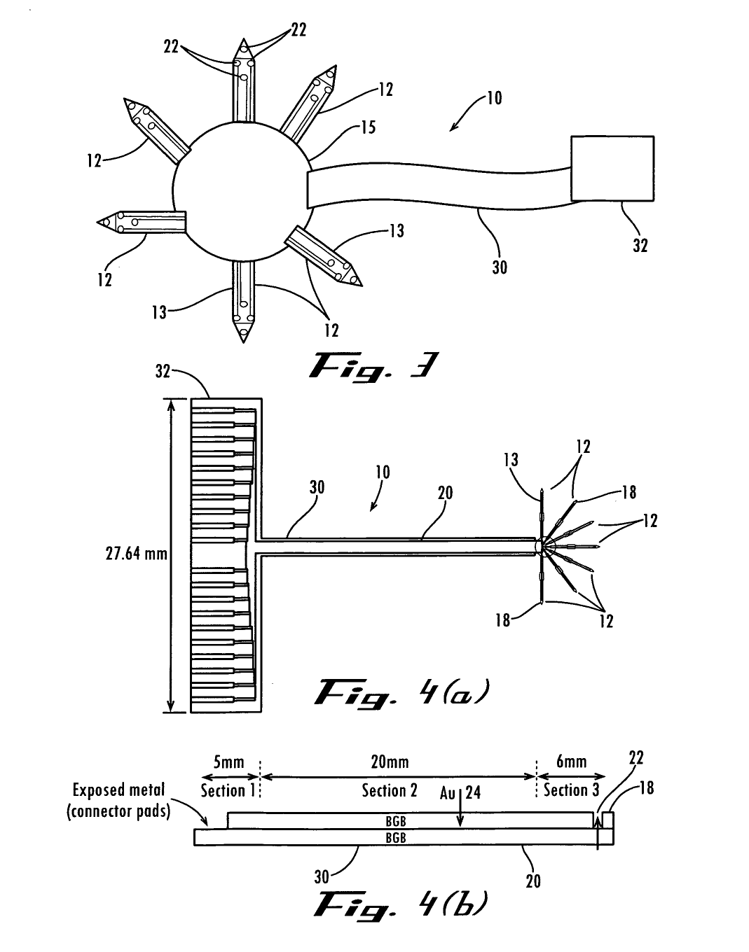

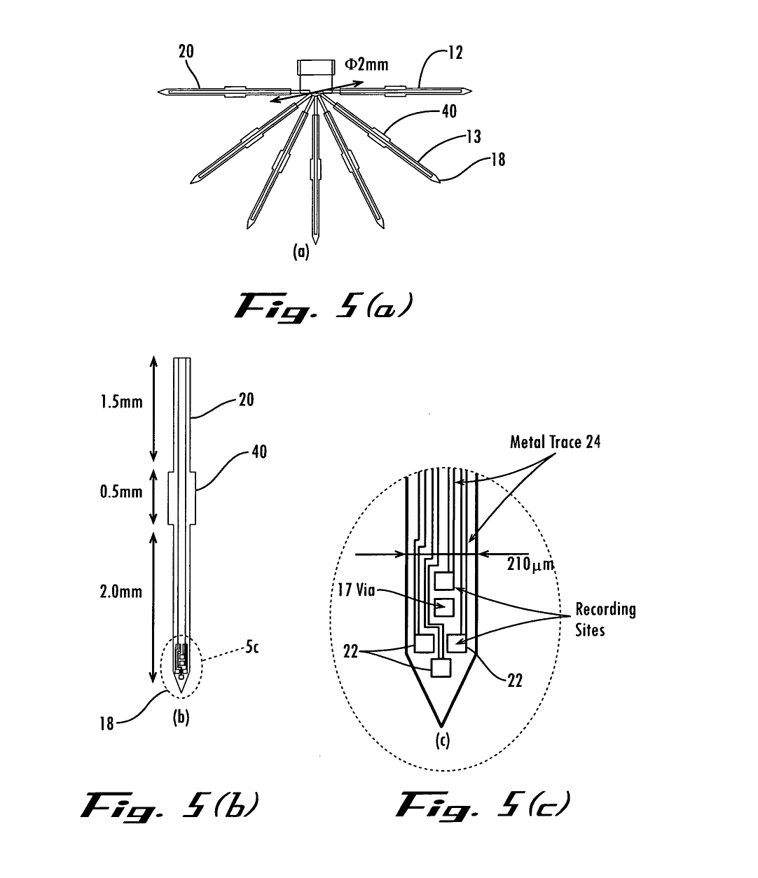

FIGS. 4(a)-4(c) show an embodiment of the intracortical electrode that is about 4 mm long and about 210 μm wide. There is one stopping and hold mark 40, which is about 310 μm wide for helping measure penetration to about 2 mm. The actual penetration or insertion portion is about 2 mm long, which is counted from distal end tip 18 to the stopping and hold mark 40. In one aspect, the portion of the electrode 12 above the stopping & hold mark improves the strength of the intracortical electrode for penetration. In this illustrated embodiment, at the tip of the intracortical electrode, as shown in FIG. 4(c), there are four transducer sites (each about 20 μm×20 μm) and one via (about 40 μm×40 μm).

FIG. 15 shows an embodiment of the intracortical electrode 12 comprising an additional silicon layer that is added underneath the tip of the electrode to stiffen it for easier penetration. In an exemplary aspect shown in Figure, the silicon backed electrode is fabricated with a 4-inch silicon-on insulator (SOI) wafer substrate with varying silicon thickness from about 10 μm and with about 1 μm thickness buried oxide. For example, the silicon can be oriented n-type silicon with resistivity of 10˜25 Ω-cm.

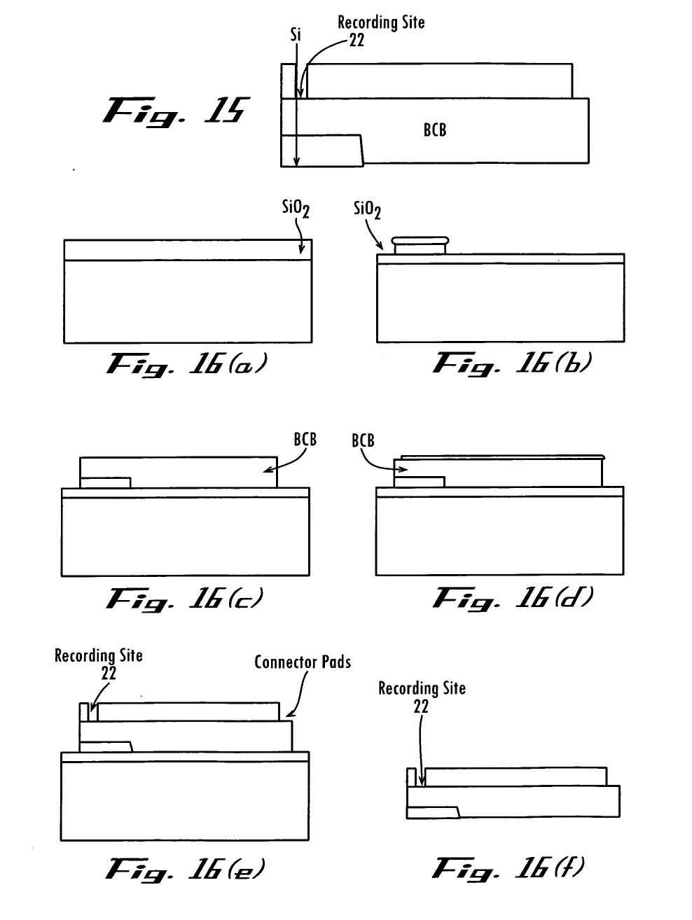

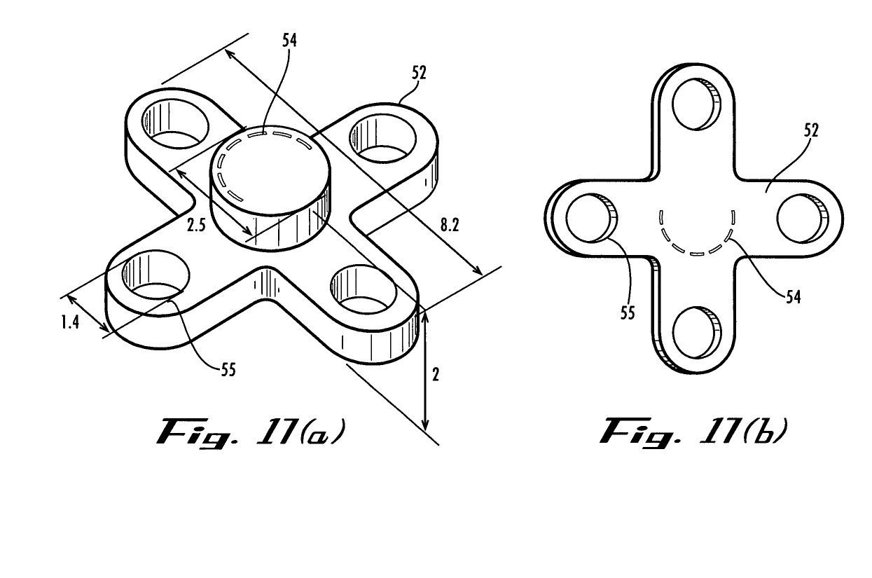

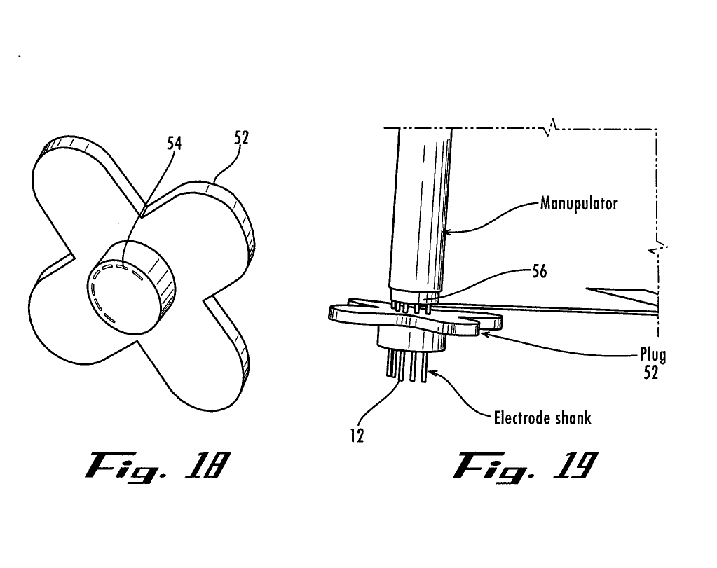

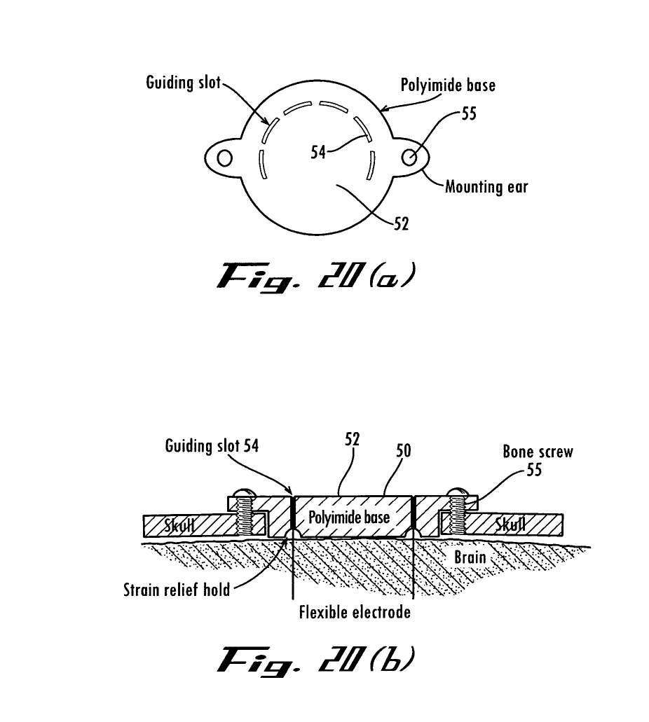

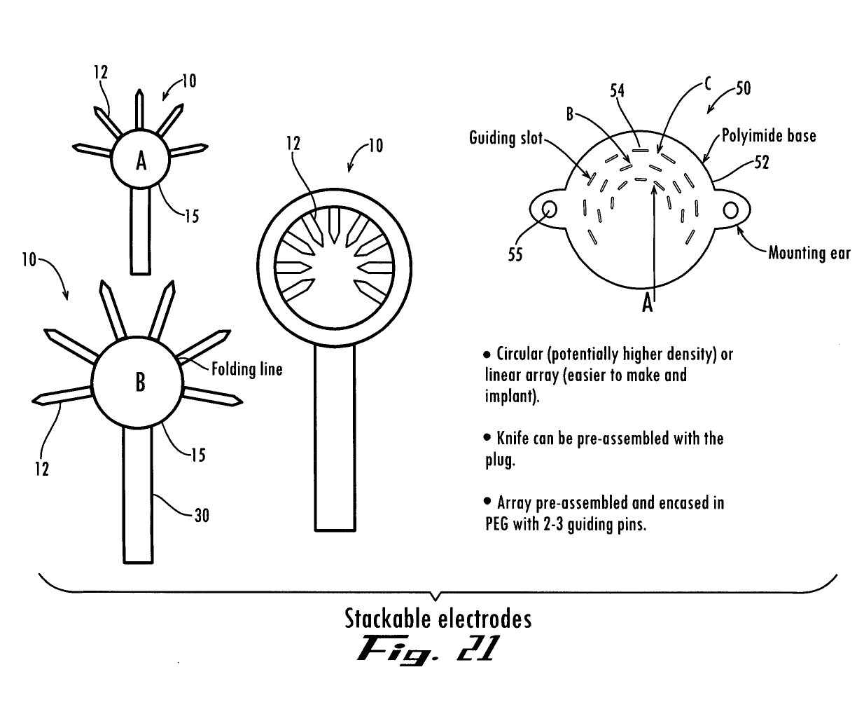

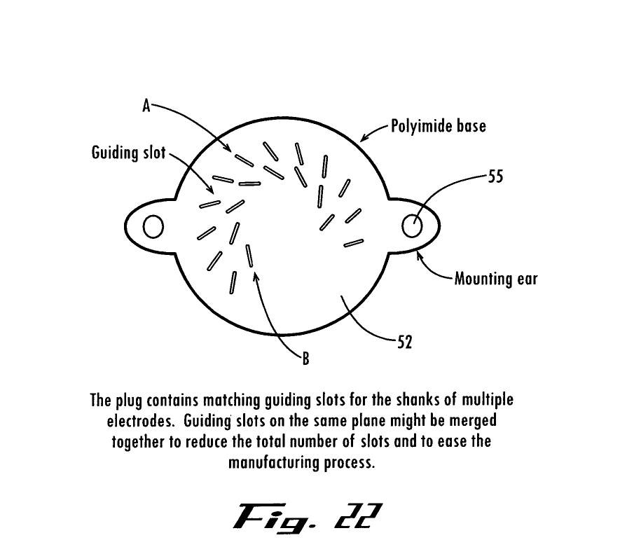

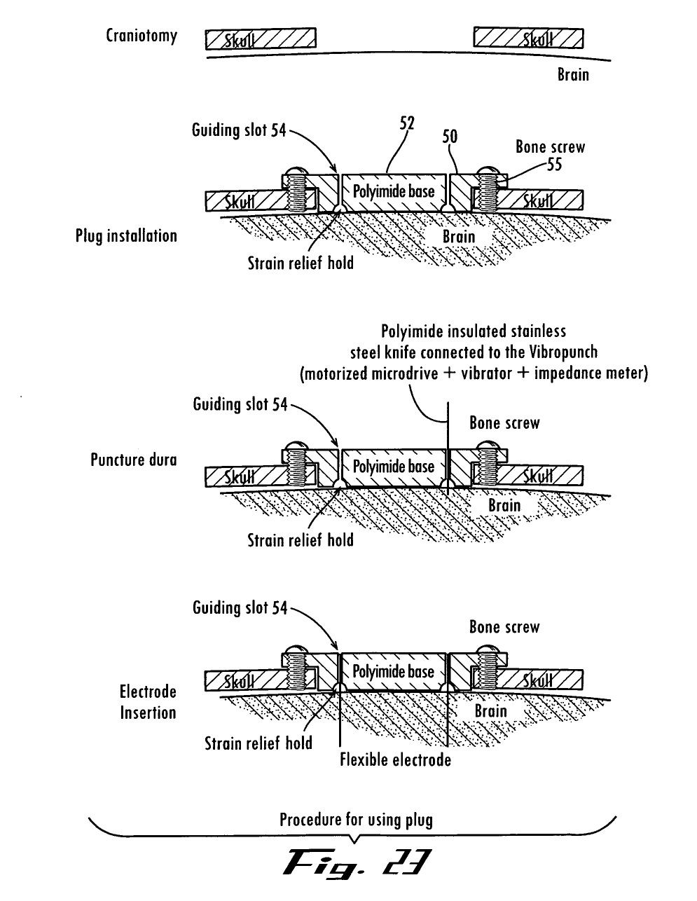

FIGS. 16(a)-16(f) show schematic diagrams for the fabrication procedure of the silicon backed electrode embodiment. The first step of the process is to define the shorter silicon backbone layer and deal with the flexible part, shown in FIG. 16(b), to make a smooth transition between flexible and stiff portions. The top silicon layer of SOI is electively etched away by using wet etching in 7% Tetra Methyl Ammonium Hydroxide (TMAH) at 80° C. The protection hard mask is 200 nm gold thin-film. The rate of silicon-etching depends on crystal planes in TMAH. See, O. Tabata, “Anisotropic Etching of Si in TMAH solutions,” Sensors and Materials, vol. 13, pp. 271-273, 2001.

The procedures shown in FIGS. 16(c)-16(f) are standard procedures as the fabrication of BCB based electrodes described herein as well as in PCT International Patent Application PCT/US2003/038027, International Publication Number WO 2004/071737, filed on 1 Dec. 2003 and US Provisional Patent Application Ser. No. 60/445,156 filed on 4 Feb. 2004, both entitled “Structure and Method of Using Benzocyclobutene as a Biocompatible Material” by He et al., both of which are incorporated herein in their entirety by reference. FIG. 16(c) shows the first layer of BCB deposition; FIG. 16(d) shows the transducer sites, metal traces and connector pads deposition; and FIG. 16(e) shows the top layer of BCB deposition with openings for the transducer site, connector pads and encapsulating the underlying metal trace. The final electrode is released from the wafer substrate by dissolving the sacrificial oxide in a 49% hydrofluoric (HF) acid solution. Several rinses with de-ionized (DI) water are then used to remove any unwanted enchant products from the released electrode. The final electrode is shown in FIG. 16(e), in which the BCB electrode gets one additional silicon layer with a shorter silicon backbone layer only on the distal end tip of the electrode.

Another embodiment of the electrode uses a sugar coating which is biocompatible and is thermo-reversible, to make the sugar coated shank temporarily stiffer than an uncoated electrode. One such sugar is glucose. When the temperature of the sugar is decreased to the freezing point, which is about 0° C., the sugar will be in a solid state. At higher temperatures, which would occur after implantation of the shank into living tissue, the sugar will melt and dissolve in the tissue's bio-fluid. The insertion portion of the tip of the electrode coated with the sugar. To solidify the glucose, before the implantation, the tip is coated by a thin layer of glucose and put into a refrigerator of at least 0° C. for about 10 hours so that the glucose is freezing and adheres to the tip tightly and stiffens the tip of the electrode 12. After implantation, the glucose will dissolve in the bio-fluid of the living tissue and the electrode will maintain its original flexibility in the tissue for minimum damage to the tissue and to comply with the brain micro-motion.

As shown in the figures, the hub member 15 provides an attachment point for the intracortical electrodes. It is contemplated that the hub member can be circular, oval, triangular, rectangular and the like, which provide the same function of providing an anchor for the shanks. In one aspect, shown in Figures, there is one hub member and seven electrodes are distributed radially around peripheral edge of the hub member with about a 27° degree separation between the respective electrodes. In one aspect the diameter of hub member is about 2 mm. For implantation, the palm covers or overlies the area of interest and the shanks are bent downwardly at a desired angle and inserted into the brain tissue.

In another aspect, the proximal end of the intracortical electrodes 12 can be connected to the hub member 15 at an angle other than tangential to a radial line extending through the center of the hub member. As previously noted, this enables the electrodes to be bent into a spiral or radial pattern relative to the center of the palm, which increasing the number of electrodes for a hub member of the same size. It can also serve to reduce the tissue damage since the minimal distance between shanks are larger for the same number of shanks.

In a further aspect, multiple assemblies 10 of increasing hub size can be arranged in an overlaying concentric pattern to future increase the electrode density.

In a further aspect, the hub member can comprise at least one reference site that is adapted to allow for differential recording to improve the signal to noise ratio. In one aspect, the at least one reference site can be positioned on the peripheral edge of the hub member.

In another aspect, at least on transponder site 22 can be positioned on the bottom surface of the hub member so that surface recording of local field potential is possible, as well as injection of signals into the neural system.

The connector 30 of the present invention can comprise a ribbon tail that provides a means to carry the electrical signals produced by the transducer sites to an interface unit 32 mounted at the distal end of the connector. In an embodiment of the invention, the ribbon tail is about 20 mm-long, but can be shorter or longer as needed for the application. When an embodiment of the assembly 10 is used in the brain, the ribbon tail will be outside the brain and can be bent to help the positioning of the interface unit. The ribbon tail can carry the electrical signals to a signal conditioning unit. In one aspect of the invention, the signal conditioning circuits can be integrated into the ribbon tail to improve the signal to noise ratio. In another embodiment, the ribbon tail is long enough to be pulled under the skin to reach signal conditioning and wireless transmission units that are implanted in other parts of the animal, such as the back or abdominal cavity.

The size of the implant assembly 10 is determined by the specific application and can vary based on the size of the subject, the implantation site, and/or the areas of interest for recording or stimulation.

Suitable insulation materials for the electrode assembly include polyimides, BCB, Parylene and other polymer materials with good mechanical, electrical, biological and thermal properties.

Suitable conductors for the electrical traces include gold, platinum, platinum/iridium or conductive polymers.

In one aspect, the electrodes 12 contain the recording or stimulating sites and can be preprocessed to be stiffer for easier penetration through the pia of the brain during surgical insertion. Each intracortical electrode 12 can be bent to an obtuse angle, for example greater than about 100°, with respect to bottom surface of the hub member as desired to position the transducer sites a specific area or depth within the cortex.

In use, the neural assemblies of the present invention are adapted to be implanted into the brain region of an animal. In one aspect, a hole or open area in the cranium is created, which is sized to accommodate the electrode assembly, and the dura in the formed craniotomy is reflected to expose the pia. The intracortical electrodes of the assembly are individually positioned and inserted into the appropriate regions of the brain with tweezers. Subsequently, the hub member is placed on top of the brain surface and the craniotomy is filled with GelForn or other bio-compatible material to seal the brain surface from outside contaminations. Next, the connector, such as the ribbon cable or tail, along with the interface unit is anchored to the surface of the skull with tissue adhesives. In one aspect, the connector and interface unit can be further secured with dental cement or surgical epoxy.

A further embodiment of the present invention comprises a guide assembly 50 that is adapted to assist with the implantation of the implant assembly 10. In one aspect, the guide assembly comprises a plug 52 that defines a plurality of guiding slots 54 that are sized and shaped to complementarily match the arrangement of the intracortical electrodes 12 extending from the implant assembly 10. The guide assembly can further comprise a guide tube 56 that is adapted to bend the electrodes and align them with the guiding slots on the plug and an inserter 58 that is adapted to independently position the electrodes and the guiding tube.

One will appreciate, and as shown in the figures, the plug 52 can be of a suitable size and shape to accommodate the intracortical electrodes of the implant assembly or assemblies. The plug 52 comprises slots 54 for each intracortical electrode to pass through as the electrode is inserted into a predetermined position in the living tissue. In another aspect, the plug can defines at least one screw holes 55, preferably on the outer edge portion of the plug such that the plug can be secured to supporting structures such as the skull. Various means for securing can be used, including adhesives and fasteners such as, for example, bone screws, micro-spikes, and the like. The plug 52 aids in limiting the exposed unsupported portion of the electrodes, which increases the maximum insertion force the shanks can exert on the brain. It also serves to prevent brain swelling, and reduces brain micro-motion.

In a further aspect, the plug 52 can comprise strain relief areas for the electrodes to reduce damage due to micromotion of the implanted tissue. The plug can be made from biocompatible materials such as polyimide, Teflon, thermal plastic, dental cement, epoxy or ceramic material.

The guiding tube 56 limits the exposed portion of the electrodes 12 during the insertion process. The diameter of guiding tube is slightly larger than the diameter of the hub member of the implant assembly. In one example, the electrodes bend inwardly into a circular pattern when the assembly is pulled through the guiding tube with a handling device, such as, for example, a nylon rod connected to the hub member of the assembly.

In one aspect, the guiding tube and the handling device are concentric and mounted on a manipulator. In a further aspect, the manipulator, the guiding tube and the handling device of the assembly are mounted on two independent lead screws so that they could be moved separately as needed.

In use, the nylon rod is first glued to the hub member of the implant assembly. It is then mounted on the inserter through the guiding tube. The electrode assembly is raised relative to the guiding tube until most of the shanks are retracted into the guiding tube. Next, the guiding tube is positioned directly above the plug that is installed in the craniotomy and secured to the skull with dental cement earlier. The exposed portions of the electrodes are then aligned with the complementary guiding slots 54 of the plug, and lowered so that the electrodes 12 go into the guiding slot and subsequently into the brain (the dura being reflected earlier). The guiding tube is raised to expose about 1 cm of nylon rod and the upper surface of the implant assembly 10. Enough dental cement is applied to embed the implant assembly, the nylon rod, and the plug to form the base of the headcap. Finally, the connector on the ribbon tail is positioned properly on the skull, and more dental cement is used to secure it and finish the headcap.

In one aspect, the dura is removed first to expose the softer pia underneath. In another aspect, rather than removing all the dura underneath the plug, the dura is kept intact before installing the plug, it is then cut open through the guiding slot to minimize tissue damage. The cutting tool is adapted to fit in the slot and cut the underlying dura without causing brain dimpling or severe damage.

One embodiment of the cutting tool is a thin flattened pin with a small hook on the tip that is about 0.1 mm thick and 0.5 mm wide. The pin is inserted at an angle through the slot till it touches the dura, once it engages the dura, it is moved along the slot to cut the underlying dura open. Another embodiment of the tool can be a laser borescope composed of a pulsed laser source and a optical fiber with micrometer focus. The fiber could be inserted through the slot to deliver the laser pulses to the dura and cut it open.

In another embodiment of the implantation device, the handling device is a hollow needle that passes through the center of the plug and the hub member. It protrudes from the center palm and is also inserted into the brain tissue when the implant assembly is lowered. The hollow needle can be used as an access port to deliver pharmacological agents to the brain. It also helps to stabilize the brain tissue around the electrodes, thus reducing the mechanical stress on the flexible electrode shanks. The length of the needle varies depending on the application.

A person skilled in the art will recognize that changes can be made in form and detail, and equivalents may be substituted for elements of the invention without departing from the scope and spirit of the invention. The present description is therefore considered in all respects to be illustrative and not restrictive, the scope of the invention being determined by the following claims and their equivalents as supported by the above disclosure and drawings.

What is claimed is:

1. An implant assembly for implantation within the neural tissue of a subject, comprising:
a hub member;
at least one electrode connected to and extending outwardly from the hub member;
a connector attached to the hub member; and
a plurality of electrical traces that extend theretrough portions of the hub member the at least one electrode and that are in communication with the connector, wherein at least a portion of each electrical trace on a portion of the electrode is exposed to form a transducer site that is adapted to convert sensed electrochemical physical reactions present in the neural tissue of the subject into electrical signals, and wherein the electrical traces are adapted to transmit the electrical signals to the connector.

2. The implant assembly of claim 1, wherein the assembly is fabricated from a flexible polymer.

3. The implant assembly of claim 2, wherein the assembly is fabricated from a thin film substrate.

4. The implant assembly of claim 3, wherein the thin film substrate is benzocyclobutene (BCB).

5. The implant assembly of claim 1, wherein the assembly further comprised means for conveying the electrical signals to an external device.

6. The implant assembly of claim 1, further comprising an external device, and wherein the connector is adapted to connect to the external device such that the electrical signals can be communicated to the external device.

7. The implant assembly of claim 6, wherein the connector comprises a ribbon cable.

8. The implant assembly of claim 7, wherein the ribbon cable is a ribbon tail fabricated of the same materials as the implant assembly.

9. The implant assembly of claim 1, wherein each shank member can be independently positioned for insertion into the location of interest.

10. The implant assembly of claim 1, wherein one or more transducers sites, with respective electrical traces, are located on the hub member.

11. The implant assembly of claim 1, wherein each electrode has a plurality of transponder sites thereon.

12. The implant assembly of claim 11, wherein the a plurality of transponder sites thereon each electrode are positioned on an insertion portion of the electrode.

13. The implant assembly of claim 1, wherein each electrode has at least one via defined thereon.

14. The implant assembly of claim 1, wherein the hub member further comprises at least one registration site that is coupled to respective electrical traces.

15. A means for recording neural signals comprising:
preparing a site on a subject for implantation of an implant assembly fabricated from a flexible polymer material;
implanting the implant assembly on the site, including insertion of intracortical electrodes from the implant assembly into the tissue of the subject; and
connecting the implant assembly to a recording device.

16. A biosensor for implanting in live tissue, comprising:
a thin film substrate including benzocyclobutene (BCB) material, wherein the thin film substrate defines an opening;
a conductor routing along the thin film substrate, wherein a portion of the conduction underlies the opening in the thin film substrate and forms a transducer site adapted to convert biophysical phenomenon to an electrical signal, and wherein the conductor is adapted to transmit the electrical signal.

17. The biosensor of claim 16, wherein the BCB material is water resistant.

18. The biosensor of claim 16, wherein the BCB material is flexible.

19. The biosensor of claim 16, wherein the BCB material is biocompatible with living tissue.

20. A method of using benzocyclobutene material in a biocompatible device, comprising the step of forming a substrate from the benzocyclobutene material so that the substrate is suitable for implant into living tissue.
